(19) United States
(12) Patent Application Publication
(10) Pub. No.: US 2018/0140220 A1
(43) Pub. Date: May 24, 2018

(54) SKIN RESISTANCE MEASURING DEVICE

(71) Applicants: KYUSHU UNIVERSITY, NATIONAL UNIVERSITY CORPORATION, Fukuoka (JP); MAZDA MOTOR CORPORATION, Hiroshima (JP)

(72) Inventors: Reiji HATTORI, Fukuoka (JP); Yuhei MORIMOTO, Fukuoka (JP); Ryota YONEDA, Fukuoka (JP); Masayuki WATANABE, Hiroshima (JP); Nanae MICHIDA, Hiroshima (JP)

(73) Assignees: KYUSHU UNIVERSITY, NATIONAL UNIVERSITY CORPORATION, Fukuoka (JP); MAZDA MOTOR CORPORATION, Hiroshima (JP)

(21) Appl. No.: 15/576,915

(22) PCT Filed: May 30, 2016
(86) PCT No.: PCT/JP2016/002607

§ 371 Date: Nov. 27, 2017

(30) Foreign Application Priority Data
May 29, 2015 (JP) 2015-110508

(51) Int. Cl. A61B 5/053; A61B 5/145

(52) U.S. Cl. CPC A61B 5/0531; A61B 5/0002; A61B 5/14517

(57) ABSTRACT

Provided herein is a skin resistance measuring device having a simple structure and capable of accurately measuring a skin resistance. An AC voltage generated by a high-frequency power source (21) is applied to a first electrode (11). A detection circuit (30) detects a current of a second electrode (12). An inductive element (25) is provided, for example, in an electric path extending from the high-frequency power source (21) to the first electrode (11). A controller (40) changes and controls a frequency of the high-frequency power source (21), receives a detection signal (S1) from the detection circuit (30), and calculates an impedance of the human body which has touched an electrode section (10). The skin resistance of the human body is calculated based on a value of impedance at a frequency where the impedance is a minimum.

### TECHNICAL FIELD

The present invention relates to a technique of measuring the skin resistance of a human body.

### BACKGROUND ART

Systems for accurately measuring the physiological information (e.g., information on the heartbeat, sweating, and breathing) of, for example, a driver inside a vehicle in a simple way at low costs, regardless of the driver's posture or conditions, have been required recently. Among such systems, those capable of measuring electrodermal activity (EDA), which is an index for electrically measuring the psychological sweating of a human body, using a simple structure have been increasingly researched and developed.

Non-Patent Document 1 shows a method of measuring EDA as skin impedance by an alternating current method using a constant current.

### CITATION LIST

### Non-Patent Documents

[Non-Patent Document 1] Hashima et al., ALTERNATING CURRENT METHOD IN ELECTRODERMAL ACTIVITY MEASUREMENT, IEICE Technical Report, Institute of Electronics, Information and Communication Engineers (IEICE), July 1995, vol. 95, No. 177, pp. 9 to 16

### SUMMARY OF THE INVENTION

### Technical Problem

However, the method disclosed in Non-Patent Document 1 requires three electrodes, and electrode paste which provides a stable impedance level for a long period of time, and greatly indicates variations in impedance for a short period. It is thus extremely difficult to apply this method to a device for measuring the physiological conditions of a driver on a daily basis.

It is an objective of the present invention to provide a skin resistance measuring device having a simple structure and capable of accurately measuring a skin resistance.

### Solution to the Problem

In order to achieve the objective, a device for measuring a skin resistance of a human body according to an aspect of the present invention includes an electrode section including a first electrode and a second electrode, and having a contact surface to be touched by the human body and covered with an insulator; a drive circuit including a high-frequency power source with a variable frequency, and configured to apply an AC voltage generated by the high-frequency power source to the first electrode; a detection circuit connected to the second electrode, and configured to detect a current of the second electrode and to output a detection signal representing a value of the detected current; an inductive element provided in an electric path extending from the high-frequency power source to the first electrode, or in an electric path extending from the second electrode to the detection circuit; and a controller configured to change and control the frequency of the high-frequency power source, and to receive the detection signal from the detection circuit. The controller calculates an impedance of the human body, which has touched the electrode section, using an output voltage of the high-frequency power source and the value of the detected current represented by the detection signal, and calculates, in a relation between the impedance and the frequency of the high-frequency power source, the skin resistance of the human body based on a value of impedance at a frequency where the impedance is a minimum.

According to this aspect, the AC voltage generated by the high-frequency power source of the drive circuit is applied to the first electrode. The detection circuit detects the current of the second electrode. In addition, the inductive element is provided in the electric path extending from the high-frequency power source to the first electrode, or in the electric path extending from the second electrode to the detection circuit. The controller changes and controls the frequency of the high-frequency power source, receives the detection signal from the detection circuit, and calculates the impedance of the human body which has touched the first and second electrodes. Then, the controller calculates the skin resistance of the human body based on the value of impedance at the frequency where the impedance is the minimum. If the human body touches here the electrode section, a capacitance is generated between the skin and the electrodes. This capacitance generates resonance together with the inductive element provided in the electric path. Thus, the controller calculates the skin resistance of the human body based on the value of impedance at the frequency where the impedance is the minimum so that the inductive element cancels the capacitances between the skin and the electrodes. This leads to accurate measurement of the skin resistance regardless of the contact state of the human body. In addition, the device requires only two electrodes and no electrode paste, and can be thus implemented by a simple structure.

In the skin resistance measuring device according to the aspect described above, the first electrode may extend in a first direction as viewed in plan, and the second electrode may include two electrodes, each located on one of two sides of the first electrode, as viewed in plan, in a second direction vertical to the first direction, and spaced apart from the first electrode.

This allows the electrode section to have the second electrode with a large surface area.

In the skin resistance measuring device according to the aspect described above, the second electrode may surround the first electrode at a predetermined distance from the first electrode, as viewed in plan.

This allows the electrode section to have the second electrode with a large surface area.

In the skin resistance measuring device according to the aspect described above, the detection circuit may include a transimpedance amplifier circuit configured to convert a current signal received from the second electrode to a voltage signal, and an envelop circuit configured to receive an output of the transimpedance amplifier circuit and to generate a signal representing an envelope waveform of the output.

In the skin resistance measuring device according to the aspect described above, when the human body touches the contact surface of the electrode section, an electric path may be formed, which passes from the first and second electrodes through the human body to earth.

In the skin resistance measuring device according to the aspect described above, a leakage resistance and a mutual capacitance may exist between the first and second electrodes.

### Advantages of the Invention

The present invention provides a skin resistance measuring device capable of accurately measuring a skin resistance regardless of the contact state of the human body using a simple structure.

### BRIEF DESCRIPTION OF THE DRAWINGS

FIG. 1 illustrates an exemplary circuit configuration of a device for measuring the skin resistance of a human body according to an embodiment.

FIG. 2 illustrates an equivalent circuit obtained by simplifying the circuit configuration of FIG. 1.

FIG. 3 is a graph showing impedance characteristics in the equivalent circuit of FIG. 2.

FIG. 4 is a revision of the equivalent circuit of FIG. 2 based on actual circumstances.

FIG. 5A is a graph showing a result of circuit simulation.

FIG. 5B is a graph showing a result of circuit simulation.

FIG. 6A is a graph showing a result of circuit simulation.

FIG. 6B is a graph showing a result of circuit simulation.

FIG. 7 illustrates an exemplary electrode design.

FIG. 8 illustrates another exemplary electrode design.

### DESCRIPTION OF EMBODIMENTS

Embodiments of the present invention will now be described with reference to the drawings.

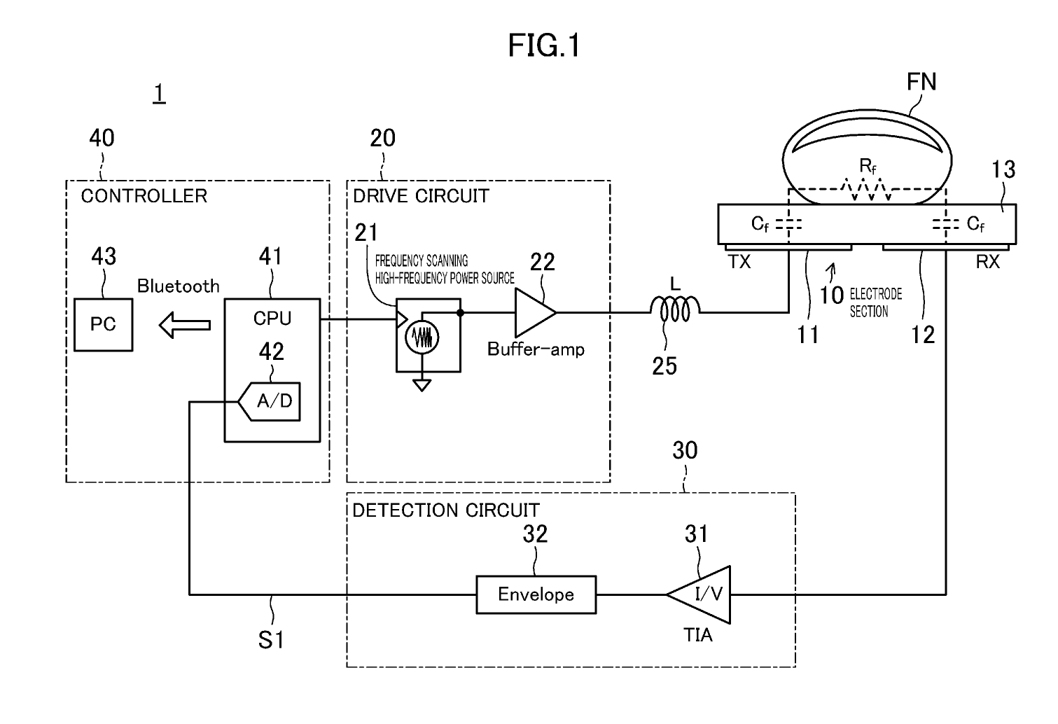

FIG. 1 illustrates an exemplary circuit configuration of a device 1 for measuring the skin resistance of a human body according to an embodiment. In FIG. 1, an electrode section 10 includes a first electrode (TX) 11 and a second electrode (RX) 12. The first and second electrodes 11 and 12 are electrically insulated from each other. The electrode section 10 has a contact surface, which is to be touched by a human body and is covered with an insulator 13. In FIG. 1 and the other figures, the surface of the electrode section 10 is to be touched by a human finger FN. Alternatively, the part touching the surface of the electrode section 10 is not limited to the finger, but may be another part of the human body.

A drive circuit 20 includes a high-frequency power source 21 with a variable frequency. The drive circuit 20 applies an AC voltage generated by the high-frequency power source 21 to the first electrode 11. The high-frequency power source 21 may be, for example, a waveform generator capable of scanning frequencies within a range from 100 kHz to 5 MHz. The drive circuit 20 includes a buffer amplifier 22, through which the AC voltage generated by the high-frequency power source 21 is applied to the first electrode 11. In an electric path extending from the high-frequency power source 21 to the first electrode 11, an inductive element 25 with an inductance L is provided.

A detection circuit 30 is connected to the second electrode 12, detects the current value of the second electrode 12, and outputs a detection signal S1 indicating the detected current value. The detection circuit 30 includes herein a transimpedance amplifier circuit 31 and an envelop circuit 32. The transimpedance amplifier circuit 31 converts a current signal received from the second electrode 12 to a voltage signal. The envelop circuit 32 receives an output from the transimpedance amplifier circuit 31, and generates a signal indicating the envelope waveform of the output. The output from the envelop circuit 32 is the detection signal S1.

A controller 40 changes and controls the frequency of the high-frequency power source 21 included in the drive circuit 20, and receives the detection signal S1 output from the detection circuit 30. The controller 40 includes here a CPU 41 and a personal computer (PC) 43. The CPU 41 includes an A/D converter 42 configured to convert the detection signals S1 to a digital signal. The PC 43 performs communications, for example, via Bluetooth (registered trademark). The CPU 41 calculates the impedance of the human body, which has touched the electrode section 10, using the output voltage of the high-frequency power source 21 and the detection signal S1 which has converted to the digital signal. Then, the CPU 41 sends the data on the impedance of the human body and the frequency of the high-frequency power source 21 to the PC 43. The PC 43 receives the data on the impedance of the human body and the frequency of the high-frequency power source 21 from the CPU 41. Then, the PC 43 calculates, in the relation between this impedance and the frequency of the high-frequency power source 21, the skin resistance R_{f} of the human body based on the value of impedance at the frequency where the impedance is the minimum.

The principle of measurement according to the present invention will now be described.

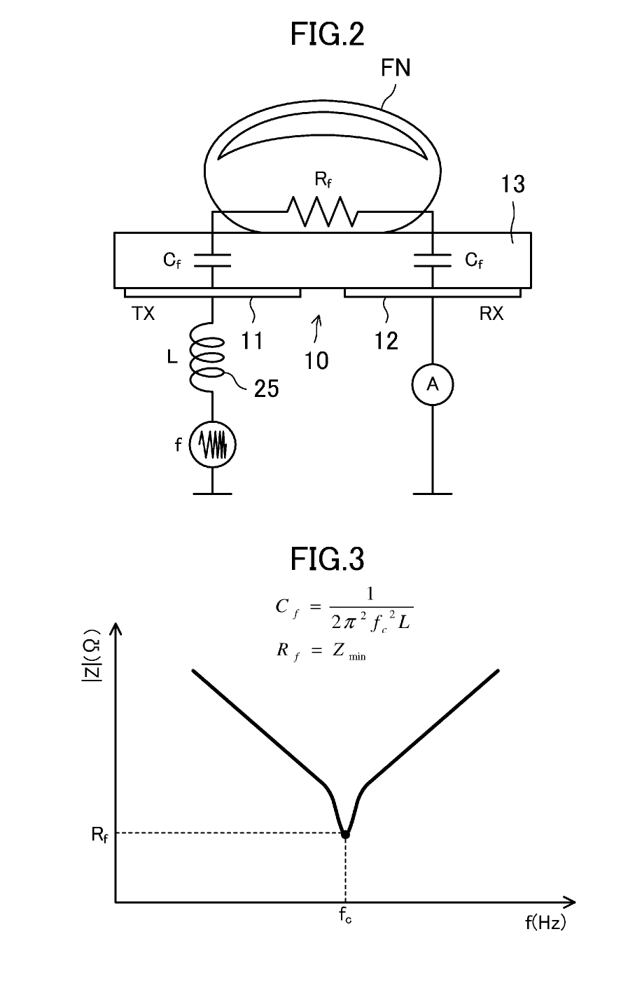

As shown in the electrode section 10 of FIG. 1, a capacitance C_{f} is generated between the human body and each of the first and second electrodes 11 and 12. For simplification, the two capacitances are equal. The circuit configuration of FIG. 1 is represented by, for example, a simplified equivalent circuit of FIG. 2. As can be seen in FIG. 2, the circuit of FIG. 1 is a series resonance circuit including the skin resistance R_{f} of the human body, the inductance L, and the capacitance C_{f}. The skin resistance measuring device 1 according to this embodiment applies an AC voltage to this resonance circuit and measures a current to measure a human body impedance Z. At this time, the human body impedance Z is represented by:

$$Z = {R}_{f} + j ( ω L - \frac{2}{ω {C}_{f}} ) \qquad ( 1 )$$

That is, the human body impedance Z includes the capacitance C_{f} between the electrodes and the human body. This capacitance C_{f} changes depending on causes such as the contact state of the human body, for example, the contact area or pressure of the finger FN when the finger FN touches the contact surface.

As shown in the graph of FIG. 3, the frequency of the AC voltage is swept to measure the impedance Z, and the impedance Zₘᵢₙ at a resonance point (a resonance frequency fc) is calculated as the resistance R_{f} of the human body. The relation between the resonance frequency f_{c} and the capacitance C_{f} is as follows.

$${C}_{f} = \frac{1}{2 {π}^{2} {f}_{c}^{2} L} \qquad ( 2 )$$

That is, in the relation between the impedance Z of the human body and the frequency of the high-frequency power source 21 obtained from the measurement result, the skin resistance R_{f} of the human body is calculated based on the value of the impedance Z at the frequency where the impedance Z is the minimum. This allows highly accurate calculation of the skin resistance R_{f} of the human body, without being affected by the capacitances between the human body and the electrodes, that is, regardless of the contact state of the human body with the electrodes.

As described above, in the present invention, the skin resistance is measured at the resonance point of impedance so that the inductive element cancels the capacitances between the skin and the electrodes. The inductive element 25 may be provided in the electric path extending from the second electrode 12 to the detection circuit 30. This case also allows measurement of the skin resistance of the human body based on the principle described above.

### Exemplary Operation

Once a user touches the electrode section 10, the skin resistance measuring device 1 starts measurement. The measurement may start at the time, for example, when a sensor recognizes that the user touches the electrode section 10, or the user executes a measurement instruction using, for example, a switch.

Once the measurement starts, the CPU 41 of the controller 40 activates the high-frequency power source 21 of the drive circuit 20. Then, the drive circuit 20 applies the AC voltage generated by the high-frequency power source 21 to the first electrode 11. The CPU 41 receives the detection signal S1 output from the detection circuit 30, while sweeping the frequency of the high-frequency power source 21. The swept frequency may fall within a range, for example, from 1 to 3 MHz. The detection signal S1 indicates the value (e.g., the maximum value or the effective value) of the AC current flowing through the second electrode 12. The CPU 41 calculates the human body impedance Z at predetermined frequencies using the output voltage of the high-frequency power source 21 and the current value indicated by the detection signal S1. Then, the CPU 41 sends each of the frequencies and the calculated impedance Z in a pair to the PC 43.

The PC 43 obtains the relation between the impedance Z and the frequency using the pairs of the impedance Z and the frequencies, which have been received from the CPU 41. Then, the PC 43 calculates the skin resistance R_{f} of the human body based on the value of impedance Z at the frequency where the impedance Z is the minimum.

As described above, in the skin resistance measuring device 1 according to this embodiment, the AC voltage generated by the high-frequency power source 21 of the drive circuit 20 is applied to the first electrode 11. The detection circuit 30 detects the current of the second electrode 12. In addition, the inductive element 25 is provided in the electric path extending from the high-frequency power source 21 to the first electrode 11, or in the electric path extending from the second electrode 12 to the detection circuit 30. The controller 40 changes and controls the frequency of the high-frequency power source 21, receives the detection signal S1 from the detection circuit 30, and calculates the impedance of the human body which has touched the electrode section 10. Then, the controller calculates the skin resistance of the human body based on the value of impedance at the frequency where the impedance is the minimum. This leads to accurate measurement of the skin resistance regardless of the contact state of the human body. In addition, the device requires only two electrodes and no electrode paste, and can be thus implemented by a simple structure.

The device configuration shown in FIG. 1 is merely illustrative, and the configurations of the circuit elements are not limited thereto. For example, the detection circuit 30 is not limited to what is shown in FIG. 1, as long as it outputs the detection signal S1 which represents the value (e.g., the effective value or the maximum value) of the high-frequency current flowing through the second electrode 12. For example, in place of the envelop circuit 32, a log amplifier or a low-pass filter may be used.

The controller 40 is not limited to the combination of the CPU 41 and the PC 43. That is, the controller 40 may have any configuration, as long as it receives the detection signal S1 from the detection circuit 30, while scanning the frequency of the high-frequency power source 21, and calculates the skin resistance R_{f} of the human body based on the value of impedance Z at the frequency where the impedance Z is the minimum.

Objective and Consideration after Experiments

The present inventors conducted experiments using a prototype of the skin resistance measuring device 1 described above. The measurement results of these experiments were, however, not as certain as expected. As a result of trial and error, the present inventors found the factor inhibiting the certainty.

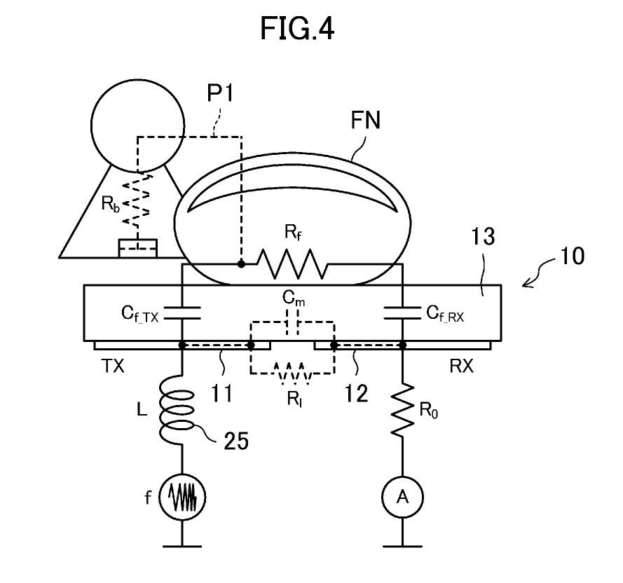

FIG. 4 is a revision of the equivalent circuit of FIG. 2 based on actual circumstances. As shown in FIG. 4, when the human body touches the contact surface of the electrode section 10, an electric path (human body-earth path) P1 is formed, which passes from the first and second electrodes 11 and 12 through the human body to earth. Even if the human body does not touch the earth, the human body-earth path P1 is formed substantially, because the human body has a large surface area. In general, the resistance R_{b} of the human body in the electric path falls within a range from about 2 to about 5 kΩ. As shown in FIG. 4, a leakage resistance R₁ and a mutual capacitance Cₘ exist between the first and second electrodes 11 and 12. The leakage resistance R₁ is a path resistance formed by a substance (e.g., sweat or dirt) attached to the surface of the electrode section 10. The mutual capacitance Cₘ is a floating capacitance within a substrate, which includes the first and second electrodes 11 and 12.

Influences of the human body-earth path P1 will now be considered. The following expression needs to be met to obtain the skin resistance R_{f} by the method described above.

R_{b}>>R_{f}+[1/j2πfC_{f}_{_}_{RX}] (3)

That is, a capacitance C_{f}_{_}_{RX} between the second electrode 12 and the human body need to be sufficiently large. For example, assume that the resistance R_{b} of the human body falls within a range 2 to 5 kΩ. In the equation of f=1 MHz, the value C_{f}_{_}_{RX} when the value of the second term on the right side is equal to the resistance R_{b} of the human body falls within a range 32 to 80 pF. Thus, C_{f}_{_}_{RX} needs to be sufficiently larger than the value. In order to obtain a capacitance of 100 pF, an insulating film with εr=10 and d=10 μm needs to have an area of about 0.1 cm².

The leakage resistance R₁ and the mutual capacitance Cₘ determine a resonance frequency f_{c0} and an impedance Z₀ at the time when the human body does not touch the contact surface. At this time, the mutual capacitance Cₘ is small, and the leakage resistance R₁ is great in a preferred embodiment so that the resonance frequency f_{c0} becomes higher than the resonance frequency f_{c} at the time when the human body touches the contact surface.

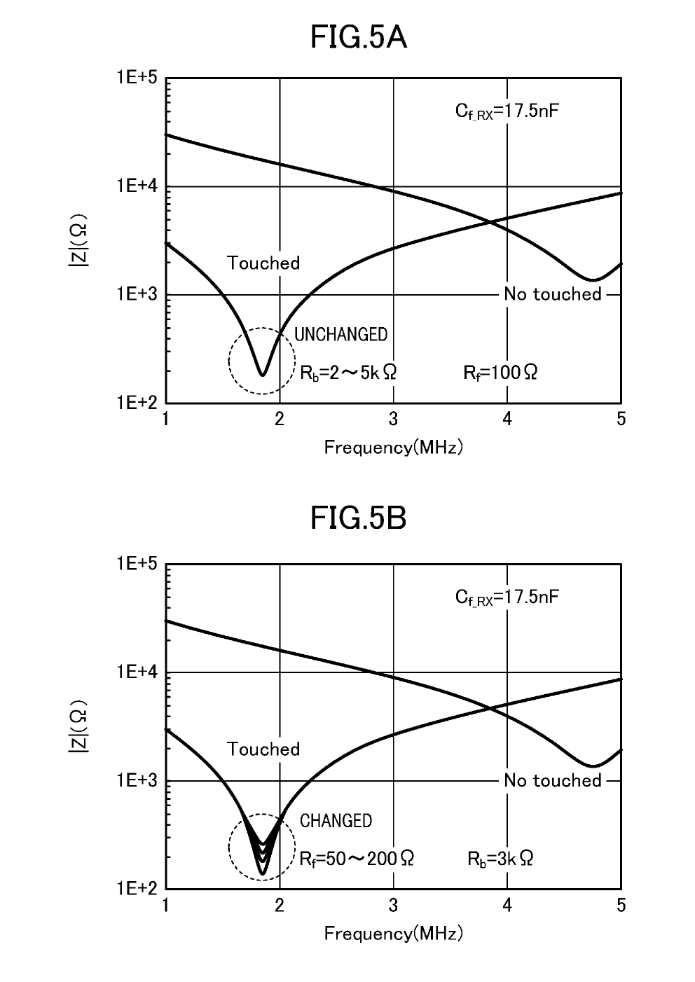

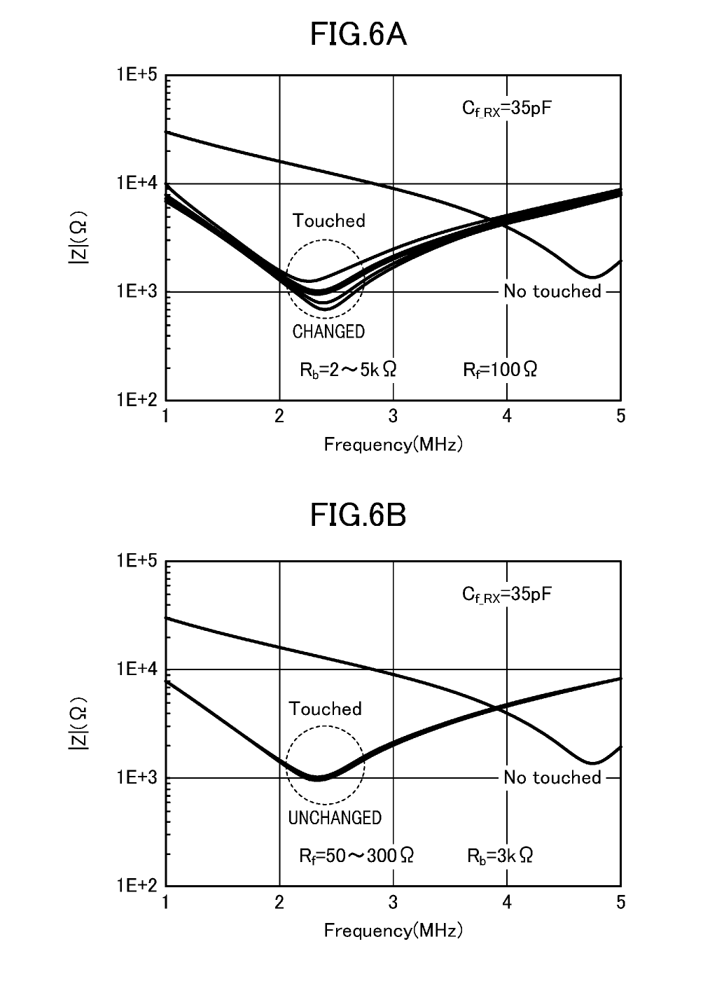

FIGS. 5A, 5B, 6A and 6B illustrate results of circuit simulation conducted by the present inventors. In FIGS. 5A and 5B, C_{f}_{_}_{RX} is equal to 17.5 nF. As shown in FIG. 5A, when R_{b} changes from 2 kΩ to 5 kΩ, the minimum value of the impedance Z does not change. On the other hand, as shown in FIG. 5B, when R_{f} changes from 50 to 200Ω, the minimum value of the impedance changes. By contrast, in FIGS. 6A and 6B, C_{f}_{_}_{RX} is equal to 35 pF. As shown in FIG. 6A, when R_{b} changes from 2 kΩ to 5 kΩ, the minimum value of the impedance Z changes. On the other hand, as shown in FIG. 6B, when R_{f} changes from 50 to 300Ω, the minimum value of the impedance does not change.

It is thus found that accurate measurement requires characterization of the configuration of the second electrode 12 so that C_{f}_{_}_{RX} is sufficiently great. On the other hand, C_{f}_{_}_{TX} does not have to be great in view of the influences of the resistance R_{b} of the human body.

### Characteristics of Electrode Configuration

Based on the consideration described above, the second electrode 12 is configured so that C_{f}_{_}_{RX} is sufficiently great. A possible configuration is, for example, as follows.

First, the insulator 13, which covers the surface of the second electrode 12, is provided with a sufficiently high dielectric constant and a sufficiently small thickness. This allows the second electrode 12 to have a great capacitance value per unit area.

Alternatively, the second electrode 12 may be provided with a sufficiently large contact area. On the other hand, the first electrode 11 does not have to have a large contact area.

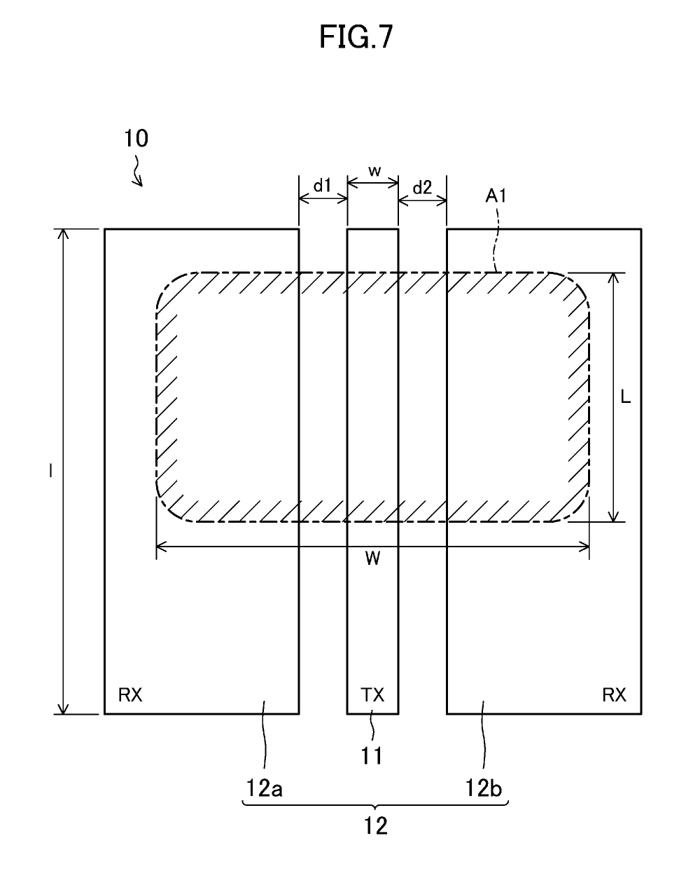

An electrode design which allows the second electrode 12 to have a large area may be employed. FIG. 7 illustrates an exemplary electrode design including the first and second electrodes 11 and 12 as viewed in plan. According to the electrode configuration of FIG. 7, the first electrode 11 extends in the vertical direction of the figure (in a first direction). The second electrode 12 includes two electrodes 12a and 12b, each of which is located on one of two sides of the first electrode 11 in the horizontal direction of the figure (a second direction vertical to the first direction), and spaced apart from the first electrode 11. The two electrodes 12a and 12b are wider than the first electrode 11, and electrically connected to each other. FIG. 7 shows a region A1, which is assumed to be touched by a human body. Employment of such an electrode design allows the second electrode 12 to have a large contact surface, without increasing the whole electrode section 10 so much. Accordingly, C_{f}_{_}_{RX} increases easily.

Assume that the first electrode 11 has a width w in the horizontal direction of the figure, and the distance between the first electrode 11 and the electrode 12a and the distance between the first electrode 11 and the electrode 12b in the horizontal direction of the figure are d1 and d2, respectively. Each of the first electrode 11 and the electrodes 12a and 12b has a length 1 in the vertical direction of the figure. The region A1 has a width W in the horizontal direction of the figure and a length L in the vertical direction of the figure. In this case, in a preferred embodiment, the expressions:

W>>w d1+d2

1>>L (4)

are met. At this time, the skin resistance R_{f} in the region A1 is inversely proportional to the length L, and the capacitance C_{f} is proportional to the length L. Since the capacitance C_{f} is calculated based on the resonance frequency f_{c}, the skin resistance R_{f} per unit contact width can be calculated regardless of the length L.

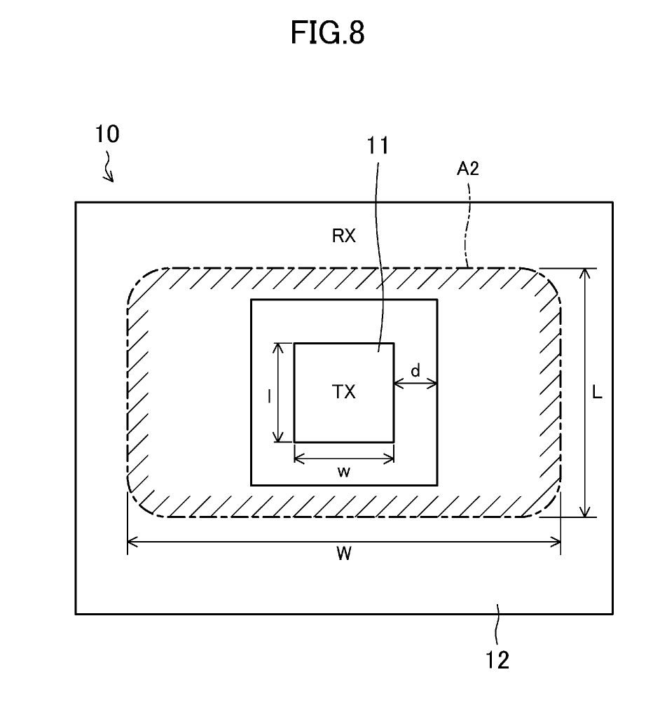

FIG. 8 illustrates another exemplary electrode design including the first and second electrodes 11 and 12 as viewed in plan. According to the electrode configuration of FIG. 8, the first electrode 11 is rectangular, and the second electrode 12 surrounds the first electrode 11 at a predetermined distance from the first electrode 11. The shape of the first electrode 11 is not limited to be rectangular, but may be, for example, circular. FIG. 8 shows a region A2, which is assumed to be touched by a human body. Employment of such an electrode design allows the second electrode 12 to have a large contact surface, without increasing the whole electrode section 10 so much. Accordingly, C_{f}_{_}_{RX} increases easily.

The first electrode 11 has a width w in the horizontal direction of the figure (a predetermined first direction), and a length 1 in the vertical direction of the figure. Assume that the distance between the first electrode 11 and the second electrode 12 is d in the horizontal direction of the figure. The region A2 has a width W in the horizontal direction of the figure, and a length L in the vertical direction of the figure. In this case, in a preferred embodiment, the expressions:

W>>w+2d

L>>1+2d (5)

are met. At this time, the skin resistance R_{f} in the region A2 is determined by the width w and the distance d regardless of the width W or the length L. The skin resistance R_{f} can be obtained regardless of the contact area.

Employment of the electrode configuration described above allows the second electrode 12 to have a large area. Accordingly, the capacitance C_{f}_{_}_{RX} between the second electrode 12 and the human body increases sufficiently, without increasing the electrode section 10.

### INDUSTRIAL APPLICABILITY

According to the present invention, a skin resistance measuring device capable of accurately measuring a skin resistance regardless of the contact state of the human body is accomplished by a simple structure. Therefore, the present invention is useful as, for example, a device for measuring, for example, the physiological information of a driver inside a vehicle.

### DESCRIPTION OF REFERENCE CHARACTERS

1 Skin resistance measuring device
10 Electrode Section
11 First Electrode
12 Second Electrode
12a, 12b Electrode
13 Insulator
20 Drive Circuit
21 High-Frequency Power Source
25 Inductive Element
30 Detection Circuit
31 Transimpedance Circuit
32 Envelop Circuit
40 Controller
S1 Detection Signal
P1 Electric Path

1. A device for measuring a skin resistance of a human body, the device comprising:
an electrode section including a first electrode and a second electrode, and having a contact surface to be touched by the human body and covered with an insulator, at least a part of the insulator which covers the second electrode having a thickness below a predetermined value;
a drive circuit including a high-frequency power source with a variable frequency, and configured to apply an AC voltage generated by the high-frequency power source to the first electrode;
a detection circuit connected to the second electrode, and configured to detect a current of the second electrode and to output a detection signal representing a value of the detected current;
an inductive element provided in an electric path extending from the high-frequency power source to the first electrode, or in an electric path extending from the second electrode to the detection circuit; and
a controller configured to change and control the frequency of the high-frequency power source, and to receive the detection signal from the detection circuit, wherein
the controller calculates an impedance of the human body, which has touched the electrode section, using an output voltage of the high-frequency power source and the value of the detected current represented by the detection signal, and calculates, in a relation between the impedance and the frequency of the high-frequency power source, the skin resistance of the human body based on only a value of impedance at a frequency where the impedance is a minimum.

2. The device of claim 1, wherein
the first electrode extends in a first direction as viewed in plan, and
the second electrode includes two electrodes, each located on one of two sides of the first electrode, as viewed in plan, in a second direction vertical to the first direction, and spaced apart from the first electrode.

3. The device of claim 1, wherein
the second electrode surrounds the first electrode at a distance from the first electrode, as viewed in plan.

4. The device of claim 1, wherein
the detection circuit includes
a transimpedance amplifier circuit configured to convert a current signal received from the second electrode to a voltage signal, and
an envelop circuit configured to receive an output of the transimpedance amplifier circuit and to generate a signal representing an envelope waveform of the output.

5. The device of claim 1, wherein
when the human body touches the contact surface of the electrode section, an electric path is formed, which passes from the first and second electrodes through the human body to earth.

6. The device of claim 1, wherein
a leakage resistance and a mutual capacitance exist between the first and second electrodes.

7. The device of claim 1, wherein
the part of the insulator which covers the second electrode has a thickness below 10 μm.
